(19) United States
(12) Patent Application Publication
(10) Pub. No.: US 2014/0275221 A1
(43) Pub. Date: Sep. 18, 2014

(54) TARGETING OF MIRNA PRECURSORS

(71) Applicant: University of Dundee, Nethergate (GB)

(72) Inventors: Angus Iain Lamond, Dundee (GB); Motoharu Ono, Dundee (GB); Kayo Ono, Dundee (GB)

(21) Appl. No.: 14/351,122

(22) PCT Filed: Oct. 10, 2012
(86) PCT No.: PCT/GB2012/052516

§ 371 Date: Apr. 10, 2014

(30) Foreign Application Priority Data
Oct. 11, 2011 (GB) 1117482.8

(51) Int. Cl. C12N 15/113

(52) U.S. Cl. CPC C12N 15/113
USPC 514/44 A; 536/24.5; 435/375; 435/320.1

(57) ABSTRACT

The present invention relates to a method of targeting mi RNA and/or premiRNA molecules in order to treat diseases that are linked with mi RNA expression, such as certain cancers. The present invention also provides modified sno RNA molecules for targeting mi RNA molecules for use in treating diseases that are linked with mi RNA expression, such as certain cancers.

### FIELD OF INVENTION

The present invention relates to a method of targeting miRNA and/or pre-miRNA molecules in order to treat diseases that are linked with miRNA expression, such as certain cancers. The present invention also provides modified snoRNA molecules for targeting miRNA molecules for use in treating diseases that are linked with miRNA expression, such as certain cancers.

### BACKGROUND TO THE INVENTION

SnoMEN (snoRNA Modulator of gene ExpressioN) technology developed by M. Ono and A. I. Lamond (1) and WO2009/037490, is a methodology for the targeted modulation of gene expression specifically for use in a gene suppression method analogous to siRNA and shRNA (FIG. 1). The major differences between snoMEN technology and other knock-down systems are a) snoMEN can be used for targeting of nuclear RNAs, e.g. pre-mRNAs and non-coding RNAs, b) snoMEN RNAs are transcribed from RNA polymerase II promoters instead of the RNA polymerase III promoter required for shRNA plasmids, c) multiple snoMEN RNAs and gene knock-in can be accomplished with a single transcript under regulation of a single promoter (FIG. 1a). These differences allow the use of snoMEN technology for a wide variety of gene regulation studies, including knock-down and/or knock-in analysis. For example, it was demonstrated that HeLa cells stably expressing GFP protein could have GFP replaced by mCherry protein using a single snoMEN plasmid vector (FIG. 1b)(1).

Cancer and other proliferative diseases (such as auto-immune disease and inflammation) are usually associated with abnormal apoptosis, or cell death. In cancer cells, for example, there is a disruption to the normal cell cycle process, which results in cancer cells being able to avoid cell death/apoptosis. One goal of many cancer treatments is to target the disruption to the normal cell cycle process and cause cancer cells to apoptose.

A number of different miRNAs have now been identified as having an association with disease development, such as cancer development. One such miRNA is mir-21. miR-21 is one of the first miRNAs detected in the human genome and displays strong evolutionary conservation across a wide range of vertebrate species in mammalian, avian and fish clades (2). The RNA expression profiles detected using high-throughput transcriptome profiling approaches for comparing miRNAs in tumours and other cell lines associated with cancer, with those of normal cells/tissues, strikingly suggested that miR-21 is over expressed in the vast majority of cancer types analysed (3). Most recently, antisense studies targeting mature miR-21 suggested that blocking miR-21 function can induce apoptosis by activating programmed cell death 4 (PDCD4) in certain types of cancer cells, e.g. HeLa cells and MCF-7 cells (4,5). While several anti-sense studies describe targeting mature miR-21 using siRNA this only affects the 22 base processed miRNA, there are no studies showing knock-down of miR-21 precursors.

### SUMMARY OF THE INVENTION

The preset invention is based on the development of modified snoRNA molecules, which are designed to target miRNA and/or pre-miRNA molecules that are involved in regulating mechanisms associated with normal cell function and hence dysregulation of miRNA has been associated with disease, such as cancer, such as the ability of normal cells to transform to a proliferative or cancer state and/or cancer cells to avoid/delay apoptosis.

In a first aspect there is provided a modified snoRNA molecule for use in a method of treating disease, such as a cancer or other disease associated with abnormal cell proliferation, the modified snoRNA molecule comprising a sequence substantially complementary to a portion of a pre-miRNA and/or miRNA molecule associated with regulation of either cancer cells, or other cells that display abnormal cell proliferation.

For the avoidance of doubt, the term “pre-miRNA” is used herein to refer to any and all forms of microRNA precessors and includes both pre-miRNA and pre-miRNA molecules referred to in the art.

As well as cancer, miRNAs and their expression have been associated with development of diseases/conditions such as cardiovascular disease, schizophrenia, renal function, Tourette's syndrome, psoriasis, primary muscular disorders, fragile-x mental retardation, polycythermia vera, diabetes, chronic hepatitis, AIDS and obesity, all of which may be the subject of the present invention.

In a preferred embodiment the disease to be treated is a cancer miRNA associated with cancer have been termed oncomir molecules and the present inventors have shown herein that it is possible to target not just the mature miRNA molecule, but also the pre-miRNA molecule. A preferred miRNA is the miRNA21 molecule (also known as hsa-mir-21) encoded by the MIR21 gene (Lagos-Quintana et al, 2001, Science 294, p 853-858). miR21 has been identified as being associated with a variety of cancers, including breast, ovarian, cervical, colon, lung, brain, oesophagus, prostrate, pancreas and thyroid and as such it may be a target for treating one or more of these cancer types. Other miRNA molecules, which may be targets of the modified snoMEN molecules of the present invention, are listed below.

miR-1 (up regulation in Cardiovascular Disease)
Let-7 family (Down regulation in breast cancer and Cardiovascular Disease)
miR-132 (up regulation in breast cancer and Cardiovascular Disease)
miR-133a (up regulation in Cardiovascular Disease)
miR-155 (up regulation in idiopathic pulmonary fibrosis (IPF))
miR-16 (down regulation in Leukemia)
miR-17˜92 cluster (down regulation in IPF/hepatocellular carcinoma)
miR-181b (up regulation in breast cancer and Cardiovascular Disease)
miR-199ab (up regulation in Cardiovascular Disease and IPF)
miR-210 (up regulation in breast cancer and cardiovascular Disease and IPF etc.)
miR-30c (down regulation in idiopathic pulmonary fibrosis (IPF))
miR-29abc (down regulation in idiopathic pulmonary fibrosis (IPF))
miR-30a-3p (down regulation in idiopathic pulmonary fibrosis (IPF))
miR30a-5p (down regulation in idiopathic pulmonary fibrosis (IPF))
miR-208 (up regulation in Cardiovascular Disease)
miR-494 (up regulation in Cardiovascular Disease/Hypoxia/Ischemia)
miR-187 (up regulation in breast cancer)
miR-340 (up regulation in breast cancer)
miR-594 (up regulation in breast cancer)
miR-31 (up regulation in colorectal cancer)
Further details of these oncomirs may be found Cho, W (2007); Mocellim et all (2009); and Esquela-Kerscher and Slack (2006) (8-10).

In a further aspect there is provided a method of modulating activity of a pre-miRNA and/or miRNA molecule associated with disease, such as cancer, the method comprising contacting the pre-miRNA and/or miRNA molecule with a snoRNA under conditions whereby the snoRNA and/or fragment thereof is capable of hybridising to a portion of the pre-miRNA and/or miRNA molecule; and wherein hybridisation of the snoRNA or fragment thereof to said portion of nucleic acid modulates activity the miRNA. The snoRNA is a modified snoRNA molecule as described further herein.

The term “modulating activity” as used herein is to be understood as reducing or inhibiting the activity of the mature miRNA by either preventing appropriate processing of the pre-miRNA molecule to the mature miRNA form and hence reducing the amount of mature miRNA, or directly binding to the mature miRNA and hence inhibiting its activity and/or altering its stability.

In terms of cancer therapy or of other diseases associated with abnormal cell proliferation, it is envisaged that the present invention may be employed in order to prevent cells which have yet to transform to a proliferative state, to transform to the proliferative or sometimes referred to as a malignant state. This may be seen as a proliferative treatment. The present invention may however be used to cause cells which have transformed to a proliferative state and are hence defective in terms of apoptosis, to now apoptose. Thus, the present invention may be used against pre-malignant and malignant cell types.

It is to be understood that modified snoRNA molecules of the present invention are not identical to native snoRNA molecules known in the art, such as the molecule HBII-180C. Generally speaking such modified snoRNA molecules are based on native snoRNA molecules, as discussed below, but comprise a portion of nucleic acid specifically selected and introduced into the snoRNA molecule, so as to hybridise to a pre-miRNA or miRNA to modulate its function.

In principle the complementary region will be of sufficient length and sequence as to provide specific binding to the pre-miRNA or miRNA molecule through known principles of complementary base pairing. Typically this may be between 15-45 nucleotides, such as 16-30 nucleotides in length.

The snoRNA molecules of the present invention may be based on so-called box C/D-snoRNA or box H/ACA-snoRNA. Preferred snoRNAs are based on box C/D-snoRNA.

As used herein, the phrase “snoRNA” refers to small RNA molecules, which usually are synthesized and/or function in the nucleoplasm and/or the nucleolus of the cell. According to the preferred embodiments the small nuclear RNA molecules of the present invention are snoRNAs that contain the box C/D.

Non-limiting examples of box C/D snoRNAs include the L. collosoma b2 (GenBank Accession No. AF331656), L. collosoma B3 (GenBank Accession No. AY046598), L. collosoma B4 (GenBank Accession No. AY046598), L. collosoma B5 (GenBank Accession No. AY046598), L. collosoma TSI (GenBank Accession No. AF331656), L. collosoma TS2 (GenBank Accession No. AF331656), L. collosoma g2 (GenBank Accession No. AF331656), L. collosoma snoRNA-2 (GenBank Accession No. AF050095), T. brucei snoRNA 92 (GenBank Accession No. Z50171, L. tarentolae snoRNA 92 (GenBank Accession No. AF016399), T. brucei TBC4 snoRNA (SEQ ID NO:35), T. brucei sno 270 (GenBank Accession No. Z50171) and human U14 snoRNA (GenBank Accession No. NRJ)00022).

The modified snoRNAs of the present invention may comprise one or more D/D′ box nucleic acid sequences commonly found in the box C/D snoRNAs. The D and/or D′ box is a conserved sequence of nucleotides and can consist of a sequence selected from, for example, 5′-CUGA-3\ 5′-AUGA-3′, 5′-CCGA-3′, 5′-CAGA-3\ 5′-CUUA-3′, 5′-UUGG-3′ and 5′-CAGC-3′. However, further modifications and/or derivatives may be envisaged.

The modified snoRNAs of the present invention may further comprise a sequence complementary to 28S rRNA and/or a Box C sequence. The sequence complementary to 28S rRNA may be from 5-15 nucleotides in length, such as 8-12 nucleotides, especially 10 nucleotides, but this rRNA complementary region can also be mutated and the complementarity to rRNA substantially or entirely removed without preventing activity. Typically the sequence complementary to 28S rRNA may be complementary to nucleotides at or around base 3680 of the 28S rRNA sequence (numbering according to Lestrade, L., and Weber, M. J. (2006). snoRNA-LBME-db, a comprehensive database of human H/ACA and C/D box snoRNAs. Nucleic Acids Res 34, D158-162.), such as around 3670-3690, e.g. 3677-3686. The box C sequence is typically 5-9 nucleotides in length, such as 7 nucleotides and may comprise the sequence AUGAUGU or a portion thereof. Typically when present a box C sequence is located 5′ of a sequence complementary either to rRNA, or to other physiological RNA targets of snoRNA, which is 5′ of a box D′ sequence, which box D′ sequence is 5′ to the nucleic acid sequence which is substantially complementary to said portion of the target nucleic acid sequence. Preferably only 1-3, such as 1 nucleotide base is found between the box C sequence, sequence complementary to rRNA, box D′ sequence and/or the nucleic acid sequence which is substantially complementary to said portion of target nucleic acid sequence. A box D sequence identical or otherwise to the box D′ sequence may be found 3′ of the nucleic acid sequence which is substantially complementary to said portion of target RNA sequence. The box D sequence may be located 20-30 nucleotides 3′ of the 3′ base of the nucleic acid sequence which is substantially complementary to said portion of target nucleic acid sequence, such as 24-28 nucleotides, especially 26 nucleotides downstream.

The modified snoRNA molecules of the present invention also comprise at least one sequence capable of targeting and hybridising to a pre-miRNA and/or miRNA molecule associated with a disease, such as cancer, as described herein.

Typically the nucleic acid sequence, which is substantially complementary to said portion of pre-miRNA and/or miRNA molecule sequence is 15-45 nucleotides in length, such as 17-30 nucleotides, but may be longer depending upon sequence and base composition. By “substantially” complementary means that there does not need to be exact complementarity between the target nucleic acid sequence and the sequence of the snoRNA molecule designed to hybridise to the target nucleic acid. However, it is to be understood that there should be a high degree of identify, typically greater than 90 or 95%, which is sufficient to ensure specific binding according to known RNA or DNA/RNA base pairing rules. Thus, typically at most only 1-3 mismatches between the two sequences may be tolerated, depending upon length and G/C content of the complementary region.

It is to be appreciated that more than one sequence designed to hybridise to a pre-miRNA and/or miRNA molecule may be found within a modified snoRNA molecule of the present invention. When there is more than one such sequence, the different “targeting” sequences may be designed to target different portions of the same pre-miRNA and/or miRNA molecule, or different pre-miRNA and/or miRNA molecules.

In a further aspect, there is also provided a nucleic acid construct capable of expressing at least one snoRNA molecule according to the present invention.

Typically the nucleic acid construct comprises at least two regions of exonic nucleic acid flanking a region of intronic nucleic acid, which is capable of encoding a snoRNA according to the present invention. Most desirably the nucleic acid construct may comprise a multiplicity of exonic sequences flanking two or more intronic sequences comprising sequence capable of encoding one or more snoRNAs according to the present invention. Such a construct may be formed as a single construct, which upon transcription within a target cell leads to generation of an mRNA corresponding to the exonic sequences and splicing out of the intronic sequence and subsequent generation of a snoRNA(s) according to the present invention.

Where more than one intronic sequence is employed to generate more than one snoRNA sequence of the present invention, each snoRNA may be designed to target the same or different pre-miRNA and/or miRNA molecules.

It is expected that the skilled addressee is well familiar with what constitutes an intron and exon, but for assistance the skilled reader is directed to WO09/037490 for a description.

The nucleic acid construct may further comprise common nucleic acid features often found in conventional nucleic acid vectors and well known to the skilled addressee. For example, the nucleic acid construct may comprise a selection marker gene for facilitating identification of cells into which the nucleic acid construct has been transformed or transfected. Preferably, the nucleic acid construct comprises at least one promoter, such as a constitutive or controllable promoter known in the art, for facilitating expression of the nucleic acid encoding said snoRNA molecule(s).

Examples of suitable promoters include the CMV promoter, T3, T7, SP6, SV40, adenovirus major late promoter and others known to the skilled addressee. In a particularly preferred embodiment of the present invention, the promoter is a regulatable RNA pol II promoter, such as rous sarcoma virus (RSV) provider (REF).

Several approaches can be used to produce the snoRNA of the present invention in a cell.

According to one preferred embodiment of the present invention the snoRNA molecule of the present invention can be produced by introducing into a cell, a nucleic acid construct capable of expressing the snoRNA molecule described above.

In order to express the snoRNA of the present invention in the cell a nucleic acid sequence is ligated into a nucleic acid construct, which includes a promoter upstream of a nucleic acid sequence encoding a snoRNA. Desirably the sequence encoding a snoRNA is flanked on either side by regions of nucleic acid identifiable by the skilled addressee as exon sequences and splicing sequences, which lead to splicing out of the sequence encoding the snoRNA upon transcription. Promoters that are suitable for directing the transcription of the nucleic acid sequence in, for example, eukaryotic cells include constitutive or inducible promoters.

Constitutive promoters suitable for use with the present invention are promoter sequences that are active under most environmental conditions and most types of cells such as the CMV promoter, SV40 promoter, adenovirus major late promoter and Rous sarcoma virus (RSV) promoter. Inducible promoters suitable for use with the present invention include for example the hypoxia-inducible factor 1 (HIF-I) promoter (Rapisarda, A. et al., 2002. Cancer Res. 62: 4316-24) and the tetracycline-inducible promoter (Srour, M. A., et al., 2003. Thromb. Haemost. 90: 398-495).

A nucleic acid construct of the present invention generally includes additional sequences that render the construct suitable for replication and/or integration in prokaryotes, eukaryotes, or preferably both (e.g. shuttle vectors). Typical cloning vectors contain transcription and translation initiation sequences (e.g., promoters, enhancers) and transcription and translation terminators (e.g., polyadenylation signals).

In the construction of the nucleic acid construct, the promoter(s) is preferably positioned at approximately the same distance from the heterologous transcription start site as it is from the transcription start site in its natural setting. As is known in the art, however, some variation in this distance can be accommodated without loss of promoter function.

In addition to the elements already described, the nucleic acid construct of the present invention may typically contain other specialised elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The nucleic acid construct may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cells, where the promoter directs expression of the desired nucleic acid. Such contracts may also comprise site-specific recombination sites, designed to target the nucleic acid construct to a specific site in a cell's genome and to integrate at the specific site when the necessary enzymes are present in the cell. A variety of site-specific recombination systems are well known to those skilled in the art, including Cre/Lox, Att/λintegrase, frt/Flp, gamma delta resolvase, Tn3 resolvase and φC231 integerase (see Gover et al., 2005, to which the skilled reader is directed).

The nucleic acid construct of the present invention can be used to express the polynucleotide of the present invention in mammalian cells (e.g., HeLa cells, Cos cells), yeast cells (e.g., AH109, HHYIO, KDY80), insect cells (e.g., Sf9), trypanosome cells (e.g., L. collosoma, L. major, T. brucei 29-13) or bacteria cells (e.g., JM109, RP437, MM509, SWIO).

Preferably, the polynucleotide of the present invention is synthesised by ligating a nucleic acid sequence into a mammalian, yeast, trypanosome or bacterial expression vector. Examples of such vectors include but are not limited to the pcDNA3.1, pBK-CMB and pCI vectors which are suitable for use in mammalian cells, the pGBKT7, pLGADH2-lacZ and pBGM18 vectors which are suitable for use in yeast cells, the pX-<<eo episomal vector which is suitable for use in trypanosome cells and the PackO2scKan, pMLBAD, pMLS7 vectors which are suitable for use in bacterial cells. According to preferred embodiments the nucleic acid construct of the present invention is preferably constructed for eukaryotic expression, most preferably, mammalian cell expression.

In accordance with an embodiment of the present invention, the vector may be a lentivirus vector known in the art, such as lenti-x expression system (Clontech), known in the art.

Examples of mammalian expression vectors include, but are not limited to, pcDNA3, pcDNA3.1(+/−), PgI3, PzEOsv2(+/−), pSecTag2, pDisplay, pEF/myc/cyto, pCMV/myc/cyto, pCR3.1, pSinRep5, DH26S, DHBB, pNMTI, pNMT41, pNMT81, which are available from Invitrogen, pCI which is available from Promega, pMbac, pPbac, pBK-RSV and pBK-CMV which are available from Stratagene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma vims include pB V-IMTHA, and vectors derived from Epstein Bar vims include pHEBO, and p205. Other exemplary vectors include pMSG, pAV009/A⁺, ρMTO10/A⁺, pMAMneo-5, baculovirus pDSVE, and any other vector allowing expression of proteins under the direction of the SV-40 early promoter, SV-40 later promoter, metallothionein promoter, murine mammary tumour vims promoter, Rous sarcoma vims promoter, polyhedron promoter, or other promoters shown effective for expression in eukaryotic cells.

Various methods can be used to introduce the nucleic acid construct of the present invention into mammalian cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, An Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al. [Biotechniques 4 (6): 504-512, 1986] and include, for example, stable or transient transfection, lipofection, electroporation, microinjection, liposomes, iontophoresis, receptor-mediated endocytosis and infection with recombinant viral vectors. In addition, see U.S. Pat. Nos. 5,464,764 and 5,487,992 for positive-negative selection methods. For example, for stable transfection in dihydrofolate reductase deficient Chinese Hamster Ovary (CHO dhfr-) cells the expression vector of the present invention further includes a dihydrofolate reductase expression cassette positioned under a control of a thymidine kinase promoter.

The nucleic acid construct of the present invention may also be delivered into a cell using viral vectors, such as lentivirus, retrovirus or adenovirus derived vectors known in the art.

In a further aspect of the present invention, there is provided a nucleic acid vector construct for use in generating a snoRNA molecule according to the present invention, the construct comprising in a 5′ to 3′ direction
i) a promoter sequence for controlling transcription;
ii) a first exon sequence;
iii) a first intron splicing sequence;
iv) a cloning site or sequence for facilitating cloning of a nucleic acid sequence encoding a snoRNA of the present invention;
v) a second intron splicing sequence; and
vi) a second exon sequence.

It is to be appreciated that the various components are transcriptionally linked as would be understood by the skilled addressee. The vector may naturally comprise other components as described herein above, and may include additional cloning sites to facilitate vector construction.

One particular advantage of the present invention is the ability to provide multiple snoRNAs from a single transcript, which can target the same or different target nucleic acid sequences.

As an alternative to the use of nucleic acid constructs, it will be appreciated that the snoRNA molecules of the present invention can be chemically synthesised using for example, solid phase synthesis, as an RNA oligonucleotide.

Several considerations must be taken into account when designing synthetic snoRNA molecules, snoRNA like molecules or fragments thereof of the present invention. For efficient in vivo inhibition of gene expression the molecules may desirably fulfil the following requirements (i) sufficient specificity in binding to the pre-miRNA and/or miRNA; (ii) solubility in water; (iii) stability against intra- and extracellular nucleases; (iv) capability of penetration through the cell membrane; and (v) when used to treat an organism, low toxicity.

Unmodified polynucleotides may be impractical for use since they have short in vivo half-lives, during which they can be degraded rapidly by nucleases. Furthermore, they are difficult to prepare in more than milligram quantities. In addition, such polynucleotides are poor cell membrane penetrants.

In order to improve half-life as well as membrane penetration, the polynucleotide backbone of the polynucleotide of the present invention can be modified.

Polynucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate bridges, carboxymethyl ester bridges, carbonate bridges, carboxymethyl ester bridges, acetamide bridges, carbamate bridges, thioether bridges, sulfoxy bridges, sulfono bridges, anomeric bridges and borane derivatives (Cook, 1991, Medicinal chemistry of antisense oligonucleotides-future opportunities. Anti-Cancer Drug Design 6: 585). Preferably, to render an in vivo stability to the synthetic polynucleotide of the present invention, the oxygen molecule at position 2 of the ribose ring may be methylated, resulting in 2′-O-methylated RNA oligonucleotides.

International patent application WO 89/12060 discloses various building blocks for synthesising polynucleotide analogs, as well as polynucleotide analogs formed by joining such building blocks in a defined sequence. The building blocks may be either “rigid” (i.e., containing a ring structure) or “flexible” (i.e., lacking a ring structure). In both cases, the building blocks contain a hydroxy group and a mercapto group, through which the building blocks are said to join to form polynucleotide analogs. The linking moiety in the oligonucleotide analogs is selected from the group consisting of sulphide (—S—), sulfoxide (—SO—), and sulfone (—SO2-).

International patent application WO 92/20702 describe an acyclic oligonucleotide which includes a peptide backbone on which any selected chemical nucleobases or analogs are strunged and serve as coding characters as they do in natural RNA. These new compounds, known as peptide nucleic acids (PNAs), are not only more stable in cells than their natural counterparts, but also bind the natural RNA 50 to 100 times more tightly than the natural nucleic acids cling to each other. PNA oligomers can be synthesised from the four protected monomers containing uridine, cytosine, adenuine and guanine by Merrifield solid-phase peptide synthesis. In order to increase solubility in water to prevent aggregation, a lysine amide group is placed at the C-terminal region.

snoRNA stability can also be increased by incorporating 3′-deoxythymidine or 2′-substituted nucleotides (substituted with, e.g., alkyl groups) into the snoRNAs during synthesis, by providing the snoRNAs as phenylisourea derivatives, or by having other molecules, such as aminoacridine or polylysine, linked to the 3′ ends of the snoRNAs (see, e.g., Anticancer Research 10:1169-1182, 1990). Modifications of the RNA nucleotides of the snoRNAs of the invention may be present throughout the snoRNA, or in selected regions, e.g., the 5′ and/or 3′ ends. The snoRNAs can also be modified to increase their ability to penetrate the target tissue by, e.g., coupling them to lipophilic compounds. The snoRNAs of the invention can be made by standard methods known in the art, including standard chemical synthesis and transcription of DNA encoding them. In addition, snoRNAs can be targeted to particular cells by coupling them to ligands specific for receptors on the cell surface of a target cell. snoRNAs can also be targeted to specific cell types by being conjugated to monoclonal antibodies that specifically bind to cell-type-specific receptors.

The nucleic acid constructs and/or vectors of the present invention may comprise more than one sequence designed to produce a modified snoRNA according to the present invention. When more than one sequence is present, the sequences may be designed to target the same target nucleic acid, or different target nucleic acids.

The nucleic acid constructs, vectors or indeed the nucleic acid encoding the modified snoRNA itself, may be designed to express further molecules such as RNAi molecules to be used in conjunction with the snoRNA molecules of the present invention, to modulate gene expression.

The same vector can be designed to encode snoRNA(s) targeted to reduce expression of a mutant gene and also encode a correct copy of the gene, within the same construct, to express the correct protein. The correct copy can be provided by way of a cDNA sequence, or alternatively be encoded by the exons, which flank the introns encoding the modified snoRNAs of the present invention. Other examples include the replacement of mutant p53 genes, BRCA genes and the like associated with cancer progression.

Advantageously, the snoRNA and replacement nucleic acid may be located within the same transcript and expressed from the same promoter as the snoRNAs that knock down expression. This has the advantage that it allows everything to be expressed as a single transcript. Moreover, this also has the advantage that it makes the expression level of the snoRNAs and the expressed replacement nucleic acid balanced and co-regulated (ie from the same promoter).

Thus, in a further aspect there is provided a snoRNA, nucleic acid, nucleic acid construct or vector according to the present invention for use in treating disease, such as a cancer or other disease associated with abnormal cell proliferation in a subject.

In a yet further aspect, there is provided a snoRNA, nucleic acid, nucleic acid construct or vector according to the present invention for use in the manufacture of a medicament for use in treating disease, such as a cancer or other disease associated with abnormal cell proliferation.

In a yet further aspect, there is provided a method of treating disease, such as a cancer or other disease associated with abnormal cell proliferation, the method comprising the step of administering to a subject in need thereof, a therapeutically effective amount of a snoRNA, nucleic acid, nucleic acid construct or vector according to the present invention.

As used herein the phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

The method according to this aspect of the present invention is effected by providing to cells of the individual the isolated polynucleotide of the present invention to thereby down regulate activity of an miRNA in the cells of the individual.

Providing can be effected by directly administering the polynucleotide of the present invention into the cells or by expressing the polynucleotide in cells as described hereinabove. Expressing can be effected by directly transfecting cells of the individual with a nucleic acid construct capable of expressing the polynucleotide of the present invention (i.e., in vivo transfection), or by transfecting cells isolated from the individual with the nucleic acid construct and administering the transfected cells to the individual (i.e., ex vivo transfection).

In a further aspect there is provided a pharmaceutical composition comprising a snoRNA according to the present invention, or a nucleic acid construct capable of expressing a snoRNA molecule according to the present invention and a pharmaceutically acceptable carrier therefore.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersable granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuff's, for example by way of an intermediate premix, or diluted in animal drinking water.

Liquid concentrates of this invention suitably contain the compound or a derivative or salt thereof and may optionally include a veterinarily acceptable water-miscible solvent, for example polyethylene glycol, propylene glycol, glycerol, glycerol formal or such a solvent mixed with up to 30% v/v of ethanol. The liquid concentrates may be administered to the drinking water of animals.

The present invention will now be further described by way of example and with reference to the figures which show:

FIG. 1: summarises features of snoMEN technology

(a) Schematic diagram showing differences between the siRNA/shRNA and snoMEN systems. Arrows show promoters for RNA polymerase III (shRNA) and RNA polymerase II (snoMEN), respectively. Grey squares show the coding region, such as mCherry cDNA or endogenous genes. White squares show non-coding Exon region. The bars show non-coding regions, e.g. introns. (b) An example of snoMEN protein replacement from GFP-SMN1 to mCherry (1). Scale bar shows 15 μm.

FIG. 2: snoMEN vector targeting the miR-21 precursor

(a) Schematic diagram showing structure of a snoMEN vector (mCherry-pre-miR-21 snoMEN) targeting the miR-21 precursor and also showing an oncogenic pathway of miR-21. Three snoMEN we targeted to different regions of the miR-21 precursor sequence. (b) Microscope images show that transiently transfection of mCherry-pre-miR-21 snoMEN induced apoptosis in HeLa cells while transfection with a plasmid encoding control snoMEN did not cause apoptosis. Scale bar shows 10 μm.

FIG. 3: Inducible stable cell line expressing pre-miR-21 snoMEN

(a) Schematic diagram shows the structure of an Inducible snoMEN vector (mCherry-pre-miR-21 inducible snoMEN) that targets the miR-21 precursor. Three snoMEN are targeted to different regions of the miR-21 precursor sequence, as shown in FIG. 2a. (b) Microscope images show examples where IPTG induction of mCherry-pre-miR-21 inducible snoMEN resulted in apoptosis in the U2OS stable cell line. Scale bar shows 10 μm.

FIG. 4: Development of a lenti-virus snoMEN vector

(a) Schematic diagram showing the structure of a lenti-virus snoMEN vector (lenti-mCherry snoMEN) that targets knock-down of G/YFP. Three snoMEN we targeted to different regions of the G/YFP sequence and expressed from an RNA that includes the mCherry cDNA sequence. All RNAs were inserted into multi cloning site (MCS) of lenti-X expression vector (Clontech). Lenti-virus was produced as described in manufacture's instructions and titrated by checking mCherry expression. The virus was transduced into WI-38 primary cells, with ˜50% transfection efficiency. Microscope images show examples of transduction with the control lenti-virus snoMEN (lenti-triple chimera targeting G/YFP) which resulted in expression of mCherry without a cytotoxic effect. Scale bar shows 10 μm. (b) Microscope images (left panel) show the result of FISH analysis after transduction of a virus encoding lenti-triple HBII-1800 snoMEN, which has the same structure as lenti-triple chimera, except snoMEN region encodes HBII-1800 snoRNA, i.e. the original backbone of snoMEN (1). Right panel shows the result of Northern blot analysis. Membrane was hybridised with radioisotope labelled RNA oligo probes to detect either HBII-1800 snoRNA or U1 snRNA as a control. (c) Microscope images show the result of transduction with viruses encoding either lenti-triple HBII-1800 (lenti HBC×3) as a control, or lenti-triple Chimera (lenti Chimera×3) to suppress GFP expression in the HeLa^{GFP} stable cell line. Lenti-Chimera×3 transduction showed GFP suppression and parallel replacement with mCherry expression (arrow). However, transduction with a control snoMEN virus didn't show suppression of GFP expression (arrow head). Scale bar shows 15 μm.

FIG. 5: Transduction of pre-miR-21 snoMEN virus in human primary and cancer cells

(a) Experimental design involves transduction of pre-miR-21 snoMEN virus into human lung primary and cancer cells. Schematic diagram shows the structure of lenti-pre-miR-21 snoMEN vector (lenti-mCherry-pre-miR-21 snoMEN), that encodes snoMEN targeting the miR-21 precursor as shown in FIG. 2a. Lenti-virus was transduced into human Lung primary cells and cancer cells. (b) Structures of lenti-pre-miR-21 snoMEN vectors. Schematic diagram shows the structures of lenti-pre-miR-21 snoMEN vectors (mCherry-pre-miR-21 snoMEN_{—}1 and _{—}2) that encode snoMEN targeting different regions of the miR-21 precursor and also shows an oncogenic pathway of miR-21.

FIG. 6: Transduction of pre-miR-21 snoMEN virus into human primary and cancer cells

(a-c) Lenti-viruses were titrated by checking mCherry expression and were transduced into human lung primary cells (left panel) and Lung cancer cells (right panel), with ˜90% transfection efficiency. Microscope images show cells 3 days after transduction with viruses encoding mCherry-pre-miR-21 snoMEN_{—}1 (a), mCherry-pre-miR-21 snoMEN_{—}2 (b), and mCherry-CM snoMEN, which targets G/YFP as a control (c), respectively. Exactly the same amount and the same batch of virus was transduced into both primary and cancer cells in each experiment. Scale bar shows 10 μm.

FIG. 7: Shows Expression analysis of pre-miR21 snoMEN. Fluorescence In Situ Hybridisation showing nucleolar localization of snoMEN targeting miR21 precursor (Cy3) expressed by transfection of mCherry-pre-miR21 snoMEN_{—}1 and mCherry-pre-miR21 snoMEN_{—}2 (FIG. 5b). DNA is stained by DAPI. Scale bar is 10 μm. Arrow shows nucleolus. Note, snoMEN show nucleolar localisation as well as Fibrillarinnucleolar protein.

FIG. 8: Shows Schematic diagram showing structure of a snoMEN vector (mCherry-pri-miR-21 snoMEN) targeting the miR-21 precursor and also showing an oncogenic pathway of miR-21. Three snoMEN we targeted to different regions of the miR-21 precursor sequence. (B) Fluorescence In Situ Hybridisation showing nucleolar localisation of snoMEN targeting miR21 precursor (Cy3). DNA is stained by DAPI. Scale bar is 10 μm. Arrow shows nucleolus. (C) MiR21 expression is suppressed by targeted snoMEN transfection. After RNA isolation, qRT-PCR (left panel) and Northern blot (right panel) analysis was performed for the levels of miR21. Graph depicts mean and standard deviation from a minimum of three independent experiments.

FIG. 9 shows Another example of snoMEN targeted knock-down to miRNA precursors. The same qRT-PCR analysis was performed as shown in Supplementary FIG. 2(C), except snoMEN targeted miRNAs are different. (B) Microscope images show that 3 days after transiently transfection of mCherry-pre-miR-21 snoMEN_{—}1/mCherry-pre-miR-21 snoMEN_{—}2/mCherry-pri-miR-21 snoMEN/mCherry-primiR31 snoMEN induced apoptosis in HeLa cells while transfection with a plasmid encoding control snoMEN did not cause apoptosis. Scale bar shows 10 μm. Note: Result shows a clear reduction of miRNA precursors by transfecting snoMEN targeted as well as miR21 targeting. Moreover, the cells were induced apoptosis (arrow) by transfecting of mCherry-pri-miR-31 snoMEN as well as miR-21 targeted snoMEN.

### MATERIALS AND METHODS

### Plasmid Construction and Transfections

The M box sequences of HBII-1800 snoMEN were modified to make them complementary to target genes as previously described (Ono et al., 2010,). The M box sequences are described below:

M box sequences of G/YFP target snoMEN, set1 5′-GACTTGAAGAAGTCGTGCTGC-3′, set2 ACCTTGATGCCGTTCTTCTGC, set3 5′-ATGATATAGACGTTGTGGCTG-3′.
M box sequences of pre-miR-21 snoMEN 1, set1 5′-TGGATGGTCAGATGAAAGATACC-3′, set2 5′-TACCCGACAAGGTGGTACAGCCA-3′, set3 5′-GCCATGAGATTCAACAGTCAA-3′.
M box sequences of pre-miR-21 snoMEN 2, set1 5′-ACATCAGTCTGATAAGCTACC-3′, set2 5′-CAGACAGCCCATCGACTGGTG-3′, set3 5′-GCCATGAGATTCAACAGTCAA-3′.
The plasmids were transfected into either HeLa cells or U2OS cells using “effectine” transfection regent (QIAGEN). SnoMEN constructs were subcloned into lenti-X expression system (Clontech) and snoMEN lenti-virus particles were produced and transduced into the cells according to the manufactures's procedures.

### Cell Culture

The human osteosarcoma U2OS cells and cervical cancer HeLa cells were grown in Dulbecco's modified Eagle's medium (DMEM) supplemented with 10% fetal bovine serum (FBS). ATCC-CCL-211, Lung Fibroblast, Human (Lung primary) cells and ATCC-CRL-5868, Lung Adenocarcinoma, Human (lung cancer) cells were purchased from ATCC and maintained in Dulbecco's modified Eagle's medium (DMEM) and RPMI 1640 supplemented with 10% fetal bovine serum (FBS), respectively.

### Microscopy

All cell images were recorded using the DeltaVision Spectris fluorescence microscope (Applied Precision). Cells were imaged using either a 10× or 60× (NA 1.4) Plan Apochromat objective. Twelve optical sections separated by 0.5 μm were recorded for each field and each exposure (SoftWoRx image processing software, Applied Precision).

### Northern RNA Blot Analysis

Total cell RNA was isolated using the TRIzol method, with DNase I treatment, according to the manufacturer's instruction (Invitrogen). Equal amounts of RNA from each sample were separated by 8M Urea polyacrylamide denaturing gel electrophoresis in 1×TBE buffer and the RNA transferred onto nylon membrane (Hybond-N; Amersham) by electro blotting. After either UV cross linking or chemical cross linking, the membrane was hybridized with ³²P 5′ end-labelled oligoribonucleotide probes specific for the following RNA species; (HBII-180C: 5′-GUGCACUGUGUCCUCAGGGGUG-3′, U1 snRNA 5′-CCACUACCACAAAUUAUGCA-3).

### Fluorescent In Situ Hybridization (FISH)

FISH procedure was performed as previously described [http://www.singerlab.org/protocols]. HeLa cells were transfected with a plasmid vector containing the HBII-1800 mini gene expressed from the CMV promoter. The cells were fixed with 4% paraformaldehyde after pre-permeabilization with 1% tritonX-100. After 70% ethanol treatment, a Cy-3 labeled HBII-1800-specific oligonucleotide probe (5′-AAAGGTCCTGGGGTGCACTGTGTCCTCAGGGGTGATCAGAGCCCAGTGCT-3′) was hybridized using standard procedures. The fluorescence signal was imaged using a Deltavision Spectris fluorescence microscope (Applied Precision).

### Results and Discussion

### Example 1

### snoMEN Targeting miR-21 Precursor can Induce Apoptosis in Transformed Cells

A snoMEN vector (mCherry-pre-miR-21 snoMEN) encoding three snoMEN in a single transcript was designed to target the precursor of human micro RNA-21 (miR-21), which is a key regulator of oncogenic processes (FIG. 2a). As described above, snoMEN technology can potentially target a wide range of nuclear RNAs using pathways separate to those regions for siRNA/shRNA activity. Therefore, we anticipated that snoMEN targeting the miR-21 precursor may be able to induce apoptosis in cancer/tumour cells.

Transient transfection of mCherry-pre-miR-21 snoMEN plasmids into either HeLa cells or U2OS cells, both of which highly express miR-21 (6), showed that the transfected cells were induced to apoptose (FIG. 2b arrow), in contrast, cells transfected with a control snoMEN plasmid (FIG. 2b arrow head) that didn't target any endogenous genes, did not apoptose. Next, we tested the expression of snoMEN from a regulatable RNA pol II promoter. For this, a plasmid was constructed that encodes mCherry-pre-miR-21 snoMEN under the control of the RSV promoter linked to the bacterial LacI operators (FIG. 3a). An inducible U2OS stable cell line was established by transfecting U2OS cells with the mCherry-pre-miR-21 inducible snoMEN plasmid. Expression of mCherry-pre-miR-21 snoMEN can be induced by adding IPTG to the culture medium. The resulting U2OS stable cells showed expression of mCherry fluorescent protein 24 hours after IPTG induction and these mCherry positive cells showed an apoptosis phenotype 36 hours after IPTG induction (FIG. 3b arrows). These results suggest that snoMEN can indeed target a microRNA precursor. This provides more flexibility in the design of knock-down vectors than targeting only, short mature miRNA sequence that has only about 22 bases.

### Example 2

### Expression from a Lenti-Virus Vector snoMEN

To improve the delivery efficiency of snoMEN, i.e. improve the transfection efficiency of snoMEN expression vectors, lenti-virus snoMEN vectors were constructed (FIG. 4a). Most mammalian cells are susceptible to lenti-virus infection, including both dividing and non-dividing cells, stem cells, and primary cells (7). The lenti-virus encoding snoMEN which targeted knock-down of GFP (Lenti-triple chimera) was transduced into WI-38 human lung fibroblast primary cells. These cells are difficult to transfect with DNA plasmids, using general transfection reagents. The result showed that lenti-virus transfected WI-38 cells express mCherry marker proteins after 48 hours and continue to grow and express mCherry for at least 3 weeks following transfection (FIG. 4a arrow). We also examined other types of primary cells, e.g. human skin fibroblast cells, human breast epithelial cells, human cortical neuron cells. In each case the snoMEN lenti-virus showed successful transfection resulting in mCherry expression in all cell types tested, consisted with previous studies (7)(data not shown). Fluorescence in situ hybridisation showed that a lenti-triple HBII-1800 snoMEN virus, which encodes three copies of snoRNA HBII-1800, express snoRNAs that accumulate in nucleoli (FIG. 4b left panel) (1). The expression of snoMEN from the viral vectors was also confirmed by northern blot analysis (FIG. 4b right panel, compare lanes 3 and 5 with lane 1). The transduction of a lenti-triple chimera virus, targeting knock-down of GFP in Hela^{GFP} stable cells showed that the same suppression of GFP expression as seen previously using DNA plasmid vectors encoding the same snoMEN (FIG. 1b and FIG. 4c arrows)(1). No GFP suppression results from transfection with the lenti-triple HBC that does not targeting GFP (FIG. 4c arrow heads). These results demonstrate that functional snoMEN can be delivered with high efficiency using lenti-virus particles in multiple type of mammalian cells.

### Example 3

### Transfection with a Pre-miR-21 snoMEN Virus Induces Apoptosis Specifically in Cancer Cells

Next, we compared lenti-viral expression of pre-miR-21 snoMEN in either normal or cancer cells derived from human tissues (FIG. 5a). Two separate lenti-mCherry-pre-miR-21 snoMEN were constructed that target different regions of the miR-21 precursor (FIG. 5b). Both pre-miR-21 snoMEN_{—}1 & _{—}2 also encode mCherry cDNA as an expression marker for transfected cells. Lenti pre-miR-21 viruses were transduced either into human lung fibroblast cells (age 20, normal) or into human lung adenocarcinoma cells established from a human patient (age 55, stage 2). The majority of both primary and cancer cells showed mCherry expression 24 hours after transfection. Primary cells transduced by either pre-miR-21 snoMEN_{—}1 or pre-miR-21 snoMEN_{—}2 kept growing and continued to express mCherry (FIG. 6a and b, left panels). However, the transduced cancer cells start showing a strong cytotoxic phenotype 3 days after transduction (FIG. 6a and b, right panels). Although this cancer cell specific cytotoxic phenotype was observed for transfection with both the pre-miR-21 snoMEN_{—}1 and _{—}2, transfection with the control snoMEN virus targeting GFP (no endogenous target) didn't show a cytotoxic phenotype in either primary or cancer cells (FIG. 6c). These results suggest that the pre-miR-21 snoMEN virus can be transduced into mammalian cells with high efficiency and induce apoptosis specifically in cancer cells that highly express miR-21. We propose that the use of snoMEN viruses that encode snoMEN targeting miRNA precursors associated with human disease phenotypes, including forms of cancer and leukaemia, can provide a novel method for clinical treatment.

The present inventors have made further constructs in accordance with the present invention and details of these are shown in Table 1:

TABLE 1
Actual example of snoMEN constructs targeted to oncomiRs

| snoMEN ID | Target oncomiR | Vector backbone | M box sequence |
|---|---|---|---|
| mCherry-pre-miR21 snoMEN_1 | precursor of miR21 | mCherry-N1 | set1 5′-TGGATGGTCAGATGAAAGATACC-3′ |
| lenti-pre-miR21 snoMEN_1 | precursor of miR21 | pLVX-puro | set2 5′-TACCCGACAAGGTGGTACAGCCA-3′ |
| | | (lenti virus) | set3 5′-GCCATGAGATTCAACAGTCAA-3′ |
| mCherry-pre-miR21 snoMEN_2 | precursor of miR21 | mCherry-N1 | set1 5′-ACATCAGTCTGATAAGCTACC-3′ |
| lenti-pre-miR21 snoMEN_2 | precursor of miR21 | pLVX-puro | set2 5′-TCAGACAGCCCATCGACTGGTG-3′ |
| | | (lenti virus) | set3 5′-GCCATGAGATTCAACAGTCAA-3′ |
| mCherry-pre-miR16-1 snoMEN | precursor of miR16-1 | mCherry-N1 | set1 5′-GCACTGCTGACATTGCTATCATAA-3′ |
| lenti-pre-miR16-1 snoMEN | precursor of miR16-1 | pLVX-puro | set2 5′-TATGGTCAACCTTACTTCAGCAGC-3′ |
| | | (lenti virus) | set3 5′-TTAATATACATTAAAACACAACTG-3′ |
| mCherry-pre-miR31 snoMEN | precursor of miR31 | mCherry-N1 | set1 5′-CTCCTCTCCAGTTCCAAGTTACAG-3′ |
| lenti-pre-miR31 snoMEN | precursor of miR31 | pLVX-puro | set2 5′-TGGCCATGGCTGCTGTCAGACAGG-3′ |
| | | (lenti virus) | set3 5′-TATGACTCTTCAGTGTTTTACTTT-3′ |
| mCherry-pre-let-7g snoMEN | precursor of let-7g | mCherry-N1 | set1 5′-CTTCAGGATGCACTTGAGACAGGA-3′ |
| lenti-pre-let-7g snoMEN | precursor of let-7g | pLVX-puro | set2 5′-CCTCAGCCTGGAATCAGGCAAAAG-3′ |
| | | (lenti virus) | set3 5′-CAGCTGGCGCGCTGTTCCTGGCAA-3′ |
| mCherry-pre-miR181b snoMEN | precursor of miR181b | mCherry-N1 | set1 5′-AATCTCTGCACAGGGAAGAGAAAG-3′ |
| lenti-pre-miR181b snoMEN | precursor of miR181b | pLVX-puro | set2 5′-CATTCATTGTTCAGTGAGCTTGTC-3′ |
| | | (lenti virus) | set3 5′-TGGTGTGTCCACCTTTGGTTTCCT-3′ |
| mCherry-pre-miR132 snoMEN | precursor of miR132 | mCherry-N1 | set1 5′-ACAGTAACAATCGAAAGCCACGGT-3′ |
| lenti-pre-miR132 snoMEN | precursor of miR132 | pLVX-puro | set2 5′-CCGGCGCGGGGCGGGCTGACGTCA-3′ |
| | | (lenti virus) | set3 5′-GCGCGTGGGCGTGCTGCGGGGCGA-3′ |
| mCherry-pre-miR210 snoMEN | precursor of miR210 | mCherry-N1 | set1 5′-GTCGCGCTGCCCAGGCACAGATCA-3′ |
| lenti-pre-miR210 snoMEN | precursor of miR210 | pLVX-puro | set2 5′-CACAGTGGGTCTGGGGCAGCGCAG-3′ |
| | | (lenti virus) | set3 5′-CTGGAGGCACTGCCGGGTGGGCGG-3′ |

### Example 4

### Knock-Down of a Number of Different Pre-miRNA and miRNA Molecules

### Material and Methods

### RNA Isolation and Quantitative RT-PCR

Total RNA was extracted using the TRIzol method, with DNase I treatment, according to manufacturer's instruction (Invitrogen). For quantitative PCR, QuantiFast SYBR green PCR kit (QIAGEN), were used to analyse samples on the Light Cycler 48011 platform (Roche). U3 snoRNA was used as a normalising gene in all experiments. Matured miR-21 expression was analysed using a qScript microRNA cDNA Synthesis Kit and PerfeCTa SYBR green SuperMix (Quanta Biosciences), PCR primers sequences:

U3
For-5′-AGAGGTAGCGTTTTCTCCTGAGCG-3′
Rev-5′-ACCACTCAGACCGCGTTCTC-3′
miR21
For-5′-TAGCTTATCAGACTGATGTTGA-3′
Rev-QScriptUniversal primer (Quanta Biosciences)
Pre-miR21
For-5′-TGTCGGGTAGCTTATCAGACT-3′
Rev-5′-TGTCAGACAGCCCATCGACTGG-3′
Pre-miR31
For-5′-CTGTAACTTGGAACTGGAGAGGAG-3′
Rev-5′-TGGCCATGGCTGCTGTCAGACAGG-3′
Pre-let-7g
For-5′-CTTTTGCCTGATTCCAGGCTGAGG-3′
Rev-5′-CAGCTGGCGCGCTGTTCCTGGCAA-3′
Pre-miR181b
For-5′-CTTTCTCTTCCCTGTGCAGAGATT-3′
Rev-5′-CATTCATTGTTCAGTGAGCTTGTC-3′
Pre-miR132
For-5′-TGACGTCAGCCCGCCCCGCGCCGG-3′
Rev-5′-GCGCGTGGGCGTGCTGCGGGGCGA-3′
Pre-miR210
For-5′-TGCGCTGCCCCAGACCCACTGTGC-3′
Rev-5′-CGGACACGGGGCCAGGAGGGTCGC-3′

### Northern Blot Analysis

Total cell RNA was isolated using the TRIzol method, with DNase I treatment, according to the manufacturer's protocol (Invitrogen). Equal amounts of RNA from each sample were separated by 8M Urea polyacrylamide denaturing gel electrophoresis in 1×TBE buffer and the RNA transferred onto nylon membrane (Hybond-N; Amersham) by electro blotting. After UV cross-linking or chemical cross-linking, the membrane was hybridized with ³²P 5′ labelled oligoRNA probes specific for miR21 gene (5′-UCAACAUCAGUCUGAUAAGCUA-3′) and tRNA (5′-UGGUGGCCCGUACGGGGAUCGA-3′).

### Results and Discussion

A plasmid mCherry-pri-miR-21 snoMEN targeting to only pri-miR21 sequence which siRNA/shRNA cannot access was constructed (Supplementary FIG. 8A). All snoMEN expressed by mCherry-pre-miR-21 snoMEN_{—}1, mCherry-pre-miR-21 snoMEN_{—}2 and mCherry-pri-miR-21 snoMEN show nucleolar localization pattern as well as Fibrillarin nucleolar protein (FIGS. 7 and 8B). Both pre-miR-21 and matured miR-21 levels were suppressed by transfecting mCherry-pri-miR-21 snoMEN plasmid (FIG. 8C). Moreover, 5 of miRNA precursors, i.e. miR-31, let-7g, miR-181b, miR-132, and miR-210, were knocked-down by transfecting targeted snoMEN plasmids (FIG. 9A). All of three miR-21 targeted snoMEN plasmids, i.e. mCherry-pre-miR-21 snoMEN_{—}1, mCherry-pre-miR-21 snoMEN_{—}2 and mCherry-pri-miR-21 snoMEN, and also snoMEN plasmid targeted miR-31 precursor induced apoptosis by transfecting into HeLa cells (FIG. 9B).

These results strongly suggest that snoMEN can target the sequence of whole miRNA precursor molecules, especially pri-miRNA specific sequences which shRNA/siRNA cannot access efficiently, and can knock-down targeted miRNAs to induce phenotype changing such as apoptosis. We foresee that snoMEN technology is a potentially very useful application of miRNA targeted knock-down at both basic research and gene therapy field in future.

### REFERENCES

1. Ono, M., Yamada, K., Avolio, F., Scott, M. S., van Koningsbruggen, S., Barton, G. J. and Lamond, A. I. (2010) Analysis of human small nucleolar RNAs (snoRNA) and the development of snoRNA modulator of gene expression vectors. Mol Biol Cell, 21, 1569-1584.
2. Selcuklu, S. D., Donoghue, M. T. and Spillane, C. (2009) miR-21 as a key regulator of oncogenic processes. Biochem Soc Trans, 37, 918-925.
3. Cheng, A. M., Byrom, M. W., Shelton, J. and Ford, L. P. (2005) Antisense inhibition of human miRNAs and indications for an involvement of miRNA in cell growth and apoptosis. Nucleic Acids Res, 33, 1290-1297.
4. Frankel, L. B., Christoffersen, N. R., Jacobsen, A., Lindow, M., Krogh, A. and Lund, A. H. (2008) Programmed cell death 4 (PDCD4) is an important functional target of the microRNA miR-21 in breast cancer cells. J Biol Chem, 283, 1026-1033.
5. Sheedy, F. J., Palsson-McDermott, E., Hennessy, E. J., Martin, C., O'Leary, J. J., Ruan, Q., Johnson, D. S., Chen, Y. and O'Neill, L. A. (2010) Negative regulation of TLR4 via targeting of the proinflammatory tumor suppressor PDCD4 by the micro RNA mi R-21. Nat Immunol, 11, 141-147.
6. Farh, K. K., Grimson, A., Jan, C., Lewis, B. P., Johnston, W. K., Lim, L. P., Burge, C. B. and Bartel, D. P. (2005) The widespread impact of mammalian MicroRNAs on mRNA repression and evolution. Science, 310, 1817-1821.
7. Coffin, R. S., Howard, M. K., Cumming, D. V., Dollery, C. M., McEwan, J., Yelton, D. M., Marber, M. S., MacLean, A. R., Brown, S. M. and Latchman, D. S. (1996) Gene delivery to the heart in vivo and to cardiac myocytes and vascular smooth muscle cells in vitro using herpes virus vectors. Gene Ther, 3, 560-566.
8. Mocellin, S., Pasquali, S., and Pilati, P., (2009) Ocomirs: From Tumor biology to molecularly targeted anticancer strategies. Mini-reviews in Medicinal Chemistry, 9, 70-80.
9. Cho, W. (2007), OncomiR: the discovery and progress of micoRNA, in oncomirs. Molecular Cancer 6, 60.
10. Esquela-Kerscher, A., and Slack, F. J., (2006). Oncomirs-microRND, with a role in cancer. Nature reviews 6, 259-269.

1. A modified snoRNA molecule comprising a nucleic acid sequence substantially complementary to a portion of a pre-miRNA and/or miRNA molecule associated with disease.

2. The modified snoRNA molecule of claim 1, wherein the disease is a cancer or other disease associated with abnormal cell proliferation.

3. The modified snoRNA molecule of claim 1, wherein the disease is selected from cardiovascular disease, schizophrenia, renal function, Tourette's syndrome, psoriasis, primary muscular disorders, fragile-x mental retardation, polycythermia vera, diabetes, chronic hepatitis, AIDS, and obesity.

4. The modified snoRNA molecule of claim 1, wherein the nucleic acid sequence is substantially complementary to miRNA21 and/or pre-miRNA21.

5. The modified snoRNA molecule of claim 1, wherein the nucleic acid sequence is substantially complementary to miRNA and/or pre-miRNA molecules selected from miR1; miR-132; miR-133a; miR155; miR-16; miR-17; miR-181b; miR-199ab; miR-210; miR-30c; miR-29abc; miR-30a-3p; miR30a-5p; miR-208; miR-494; miR-187; miR-340; miR-594; and miR-31.

6. A method of reducing or inhibiting activity of a pre-miRNA and/or miRNA molecule associated with disease, comprising contacting the modified snoRNA molecule of claim 1 under conditions whereby the snoRNA molecule and/or fragment thereof is capable of hybridising to a portion of the pre-miRNA and/or miRNA molecule; and wherein hybridisation of the modified snoRNA or fragment thereof to said portion of nucleic acid reduces processing of the pre-miRNA to mature miRNA or directly binds mature miRNA and inhibits miRNA activity.

7. The modified snoRNA molecule according to claim 1, wherein the complementary region is 15-45 nucleotides in length.

8. A nucleic acid construct capable of expressing at least one snoRNA molecule according to claim 1 upon insertion into an appropriate host.

9. The nucleic acid construct of claim 8, wherein the nucleic acid construct is a lentivirus, retrovirus, adenovirus, or adeno-associated virus vector.

10. The modified snoRNA molecule of claim 1, wherein the modified snoRNA itself, is designed to express one or more further molecules to be used in conjunction with the snoRNA molecules.

11. The modified snoRNA molecule of claim 1, wherein the modified snoRNA is designed to encode snoRNA(s) targeted to reduce expression of a mutant gene and further encodes a correct copy of the gene, within the same construct, to express the correct protein.

12. A pharmaceutical composition comprising a snoRNA molecule according to claim 1 and optionally a pharmaceutically acceptable carrier therefor.

13. A method for inducing apoptosis in a cancer cell or other cell displaying abnormal cell proliferation, comprising introducing the modified snoRNA molecule of claim 1 into the cell.

14. The modified snoRNA molecule of claim 10, wherein the one or more further molecules are RNAi molecules.

15. A method for inducing apoptosis, comprising reducing or inhibiting a miR precursor in a cancer cell or other cell displaying abnormal cell proliferation.

16. The method of claim 15, wherein said miR precursor is a pre-miRNA or miRNA.

17. The method of claim 15, wherein reducing or inhibiting comprises introducing a modified snoRNA molecule comprising a sequence substantially complementary to a portion of a pre-miRNA and/or miRNA molecule associated with disease.


SEQUENCE LISTING (53 sequences)

SEQ ID NO: 1 - DNA, 21 bp, Homo sapiens
gacttgaagaagtcgtgctgc

SEQ ID NO: 2 - DNA, 21 bp, Homo sapiens
accttgatgccgttcttctgc

SEQ ID NO: 3 - DNA, 21 bp, Homo sapiens
atgatatagacgttgtggctg

SEQ ID NO: 4 - DNA, 23 bp, Homo sapiens
tggatggtcagatgaaagatacc

SEQ ID NO: 5 - DNA, 23 bp, Homo sapiens
tacccgacaaggtggtacagcca

SEQ ID NO: 6 - DNA, 21 bp, Homo sapiens
gccatgagattcaacagtcaa

SEQ ID NO: 7 - DNA, 21 bp, Homo sapiens
acatcagtctgataagctacc

SEQ ID NO: 8 - DNA, 21 bp, Homo sapiens
cagacagcccatcgactggtg

SEQ ID NO: 9 - DNA, 21 bp, Homo sapiens
gccatgagattcaacagtcaa

SEQ ID NO: 10 - RNA, 22 nt, Homo sapiens
gugcacuguguccucaggggug

SEQ ID NO: 11 - RNA, 20 nt, Homo sapiens
ccacuaccacaaauuaugca

SEQ ID NO: 12 - DNA, 50 bp, Homo sapiens
aaaggtcctggggtgcactgtgtcctcaggggtgatcagagcccagtgct

SEQ ID NO: 13 - DNA, 23 bp, Homo sapiens
tggatggtcagatgaaagatacc

SEQ ID NO: 14 - DNA, 23 bp, Homo sapiens
tacccgacaaggtggtacagcca

SEQ ID NO: 15 - DNA, 21 bp, Homo sapiens
gccatgagattcaacagtcaa

SEQ ID NO: 16 - DNA, 21 bp, Homo sapiens
acatcagtctgataagctacc

SEQ ID NO: 17 - DNA, 22 bp, Homo sapiens
tcagacagcccatcgactggtg

SEQ ID NO: 18 - DNA, 21 bp, Homo sapiens
gccatgagattcaacagtcaa

SEQ ID NO: 19 - DNA, 24 bp, Homo sapiens
gcactgctgacattgctatcataa

SEQ ID NO: 20 - DNA, 24 bp, Homo sapiens
tatggtcaaccttacttcagcagc

SEQ ID NO: 21 - DNA, 24 bp, Homo sapiens
ttaatatacattaaaacacaactg

SEQ ID NO: 22 - DNA, 24 bp, Homo sapiens
ctcctctccagttccaagttacag

SEQ ID NO: 23 - DNA, 24 bp, Homo sapiens
tggccatggctgctgtcagacagg

SEQ ID NO: 24 - DNA, 24 bp, Homo sapiens
tatgactcttcagtgttttacttt

SEQ ID NO: 25 - DNA, 24 bp, Homo sapiens
cttcaggatgcacttgagacagga

SEQ ID NO: 26 - DNA, 24 bp, Homo sapiens
cctcagcctggaatcaggcaaaag

SEQ ID NO: 27 - DNA, 24 bp, Homo sapiens
cagctggcgcgctgttcctggcaa

SEQ ID NO: 28 - DNA, 24 bp, Homo sapiens
aatctctgcacagggaagagaaag

SEQ ID NO: 29 - DNA, 24 bp, Homo sapiens
cattcattgttcagtgagcttgtc

SEQ ID NO: 30 - DNA, 24 bp, Homo sapiens
tggtgtgtccacctttggtttcct

SEQ ID NO: 31 - DNA, 24 bp, Homo sapiens
acagtaacaatcgaaagccacggt

SEQ ID NO: 32 - DNA, 24 bp, Homo sapiens
ccggcgcggggcgggctgacgtca

SEQ ID NO: 33 - DNA, 24 bp, Homo sapiens
gcgcgtgggcgtgctgcggggcga

SEQ ID NO: 34 - DNA, 24 bp, Homo sapiens
gtcgcgctgcccaggcacagatca

SEQ ID NO: 35 - DNA, 24 bp, Homo sapiens
cacagtgggtctggggcagcgcag

SEQ ID NO: 36 - DNA, 24 bp, Homo sapiens
ctggaggcactgccgggtgggcgg

SEQ ID NO: 37 - DNA, 24 bp, Homo sapiens
agaggtagcgttttctcctgagcg

SEQ ID NO: 38 - DNA, 20 bp, Homo sapiens
accactcagaccgcgttctc

SEQ ID NO: 39 - DNA, 22 bp, Homo sapiens
tagcttatcagactgatgttga

SEQ ID NO: 40 - DNA, 21 bp, Homo sapiens
tgtcgggtagcttatcagact

SEQ ID NO: 41 - DNA, 22 bp, Homo sapiens
tgtcagacagcccatcgactgg

SEQ ID NO: 42 - DNA, 24 bp, Homo sapiens
ctgtaacttggaactggagaggag

SEQ ID NO: 43 - DNA, 24 bp, Homo sapiens
tggccatggctgctgtcagacagg

SEQ ID NO: 44 - DNA, 24 bp, Homo sapiens
cttttgcctgattccaggctgagg

SEQ ID NO: 45 - DNA, 24 bp, Homo sapiens
cagctggcgcgctgttcctggcaa

SEQ ID NO: 46 - DNA, 24 bp, Homo sapiens
ctttctcttccctgtgcagagatt

SEQ ID NO: 47 - DNA, 24 bp, Homo sapiens
cattcattgttcagtgagcttgtc

SEQ ID NO: 48 - DNA, 24 bp, Homo sapiens
tgacgtcagcccgccccgcgccgg

SEQ ID NO: 49 - DNA, 24 bp, Homo sapiens
gcgcgtgggcgtgctgcggggcga

SEQ ID NO: 50 - DNA, 24 bp, Homo sapiens
tgcgctgccccagacccactgtgc

SEQ ID NO: 51 - DNA, 24 bp, Homo sapiens
cggacacggggccaggagggtcgc

SEQ ID NO: 52 - RNA, 22 nt, Homo sapiens
ucaacaucagucugauaagcua

SEQ ID NO: 53 - RNA, 22 nt, Homo sapiens
ugguggcccguacggggaucga